(19) United States
(12) Patent Application Publication
(10) Pub. No.: US 2019/0316073 A1
(43) Pub. Date: Oct. 17, 2019

(54) FERMENTATION TANK

(71) Applicants: Chin-Hsing Chuo, Taichung City (TW); Chih-Meng Wang, Taichung City (TW); Chin-Yen Wang, Taichung City (TW)

(72) Inventors: Chin-Hsing Chuo, Taichung City (TW); Chih-Meng Wang, Taichung City (TW); Chin-Yen Wang, Taichung City (TW)

(21) Appl. No.: 16/347,773

(22) PCT Filed: Dec. 9, 2016
(86) PCT No.: PCT/CN2016/109126

§ 371 Date: May 6, 2019

(51) Int. Cl. C12M 1/02; C12M 1/06; C12M 1/26; C12M 1/00

(52) U.S. Cl. CPC C12M 41/22; C12M 27/18; C12M 33/00; C12M 27/06

(57) ABSTRACT

A fermentation tank includes a tank body, enclosing a cavity therein, having a material inlet and a material outlet; a heat exchange structure, disposed on a wall of the cavity for heat exchanging with the tank body; at least one flow disturbing plate, being an elongated plate, the width direction of the flow disturbing plate being parallel to a radial direction of the tank body, the longitudinal direction of the flow disturbing plate being parallel to a vertical direction, a length of the flow disturbing plate being larger than a width thereof, the flow disturbing plate being arranged in the cavity and connected to the tank body, the flow disturbing plate having a heat exchange channel therein, two ends of the heat exchange channel communicating an exterior respectively so that heat is exchanged between the at least one flow disturbing plate and the cavity.

### BACKGROUND OF THE INVENTION

### Field of the Invention

The present invention relates to a fermentation tank.

### Description of the Prior Art

The heating system of conventional fermentation tanks includes helix pipe around the outer surface of the tank for steam or hot water to passing through or helix pipe placed in the tank. The helix pipe around the outer surface of the tank has gap between every turns. The gap doesn't distribute to heating, so the heating is not even.

The pipe in the tank has the similar problem. In addition, the pipe is located at the bottom of the tank and is distant from the wall of the tank. Thus, the tank is difficult to wash and clean.

### SUMMARY OF THE INVENTION

The main object of the present invention is to provide a fermentation tank having improved heat exchange mechanism which is easy to wash.

To achieve the above and other objects, the fermentation tank of the present invention includes a tank body, a heat exchange structure, and at least one flow disturbing plate.

The tank body has a top end and a bottom end and encloses a cavity therein. A vertical direction is defined from a line composed of the top end and the bottom end. The tank body has a material inlet and a material outlet. The heat exchange structure is disposed on a wall of the cavity for heat exchanging with the tank body. The at least one flow disturbing plate is an elongated plate and has a width direction, a longitudinal direction, and a thickness direction which are perpendicular to each other. The width direction is parallel to a radial direction of the tank body. The longitudinal direction is parallel to the vertical direction. A length of the at least one flow disturbing plate is larger than a width thereof. The at least flow disturbing plate is arranged in the cavity and is connected to the tank body. The at least one flow disturbing plate has a heat exchange channel therein. Two ends of the heat exchange channel communicate an exterior respectively so that heat is exchanged between the at least one flow disturbing plate and the cavity.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of the present invention;

FIG. 2 is a profile of the present invention;

FIG. 3A is a profile showing a flow disturbing plate of a second embodiment of the present invention;

FIG. 3B is a profile showing a flow disturbing plate of a third embodiment of the present invention;

FIG. 4 is a profile of the present invention at an other angle.

### DETAILED DESCRIPTION OF THE PREFERRED EMBODIMENTS

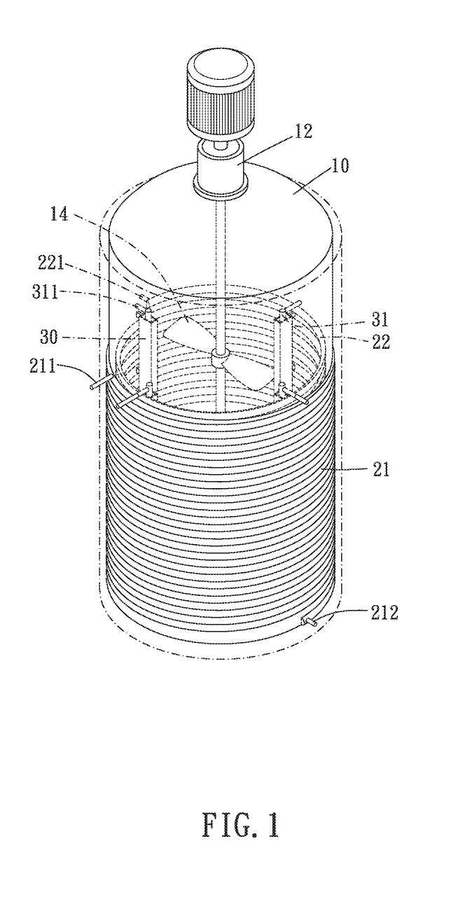

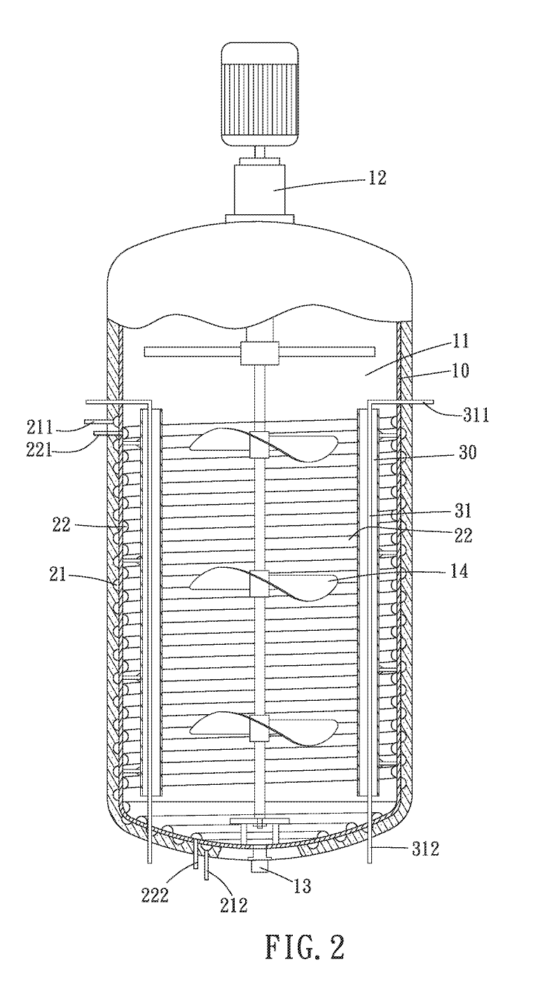

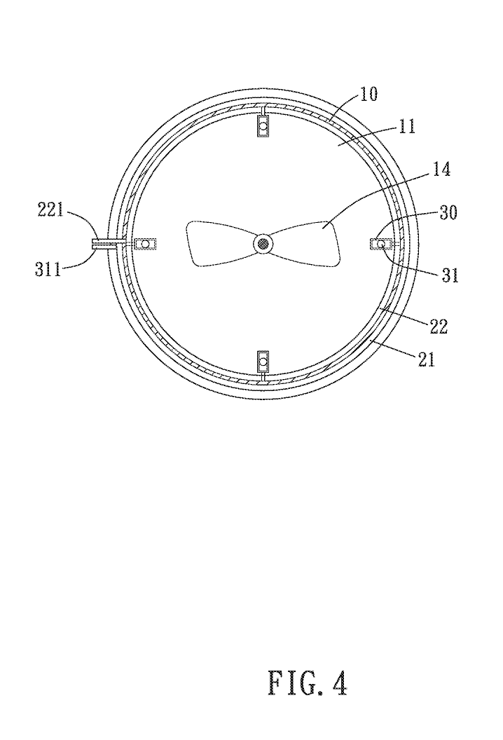

Please refer to FIG. 1 to FIG. 4, the fermentation tank of the present invention includes a tank body 10, a heat exchange structure, and at least one flow disturbing plate 30.

The tank body 10 has a top end and a bottom end and encloses a cavity 11 therein. A vertical direction is defined from a line composed of the top end and the bottom end. The tank body 10 has a material inlet 12 and a material outlet 13. The heat exchange structure is disposed on a wall of the cavity 11 for heat exchanging with the tank body 10. The at least one flow disturbing plate 30 is an elongated plate and has a width direction, a longitudinal direction, and a thickness direction which are perpendicular to each other. The width direction is parallel to a radial direction of the tank body 10. The longitudinal direction is parallel to the vertical direction. A length of the at least one flow disturbing plate 30 is larger than a width thereof. The at least flow disturbing plate 30 is arranged in the cavity 11 and is connected to the tank body 10. The at least one flow disturbing plate 30 has a heat exchange channel 31 therein. (The heat exchange channel can be directly formed in the hollow flow disturbing plate.) Two ends of the heat exchange channel 31 communicate an exterior respectively so that heat is exchanged between the at least one flow disturbing plate 30 and the cavity 11. In the present embodiment, four flow disturbing plate 30 are included and arranged spacedly.

In the present embodiment, the heat exchange structure includes an exterior pipe 21. The exterior pipe 21 extends as helix around the outer surface of the tank body 10. Any adjacent turns of the exterior pipe 21 are spacedly arranged to form a gap. The exterior pipe 21 has a first inlet 211 and a first outlet 212. The first inlet 211 and the first outlet 212 communicate the exterior respectively. Preferably, the heat exchange structure further includes an interior pipe 22. The interior pipe 22 extends as helix around the inner wall of the tank body 10. The interior pipe 22 positionally corresponds to the gaps of the exterior pipe 21. The interior pipe 22 has a second inlet 221 and a second outlet 222. The second inlet 221 and the second outlet 222 communicate the exterior respectively. Specifically, the first inlet is located at the top of the exterior pipe, and the first outlet is located at the bottom of the exterior pipe. However, in other possible embodiments, the first inlet can be located at the bottom of the exterior pipe, and the first outlet can be located at the top of the exterior pipe.

More specifically, the heat exchange channel 31 extends along the longitudinal direction and has a third outlet 311 and the third inlet 312 communicating the exterior respectively. The third outlet 311 is located at the top of the heat exchange channel 31, and the third inlet 312 is located at the bottom of the heat exchange channel 31.

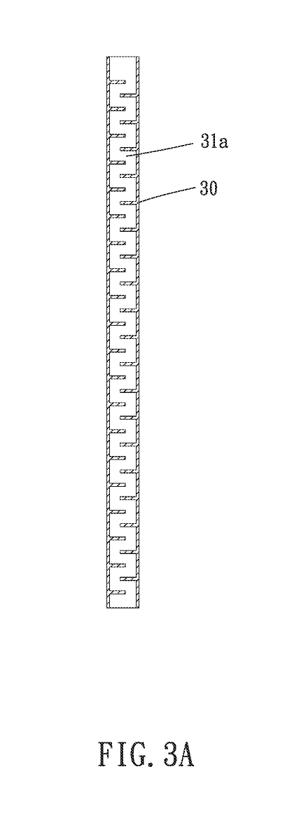

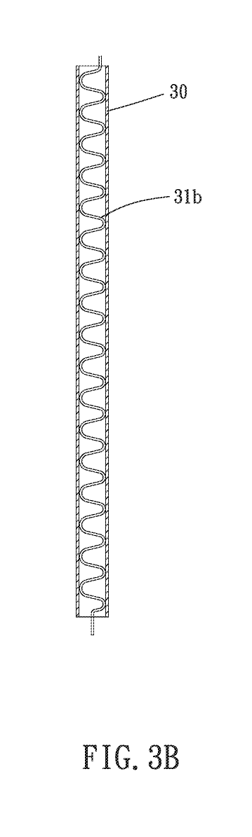

In other possible embodiments, the flow disturbing plate 30 has plural horizontal plates arranged staggered to enclose the meandering heat exchange channel 31a, as shown in FIG. 3A. Or the heat exchange channel 31b can be a meandering or helix pipe received in the flow disturbing plate 30.

Optionally, the tank body 10 has a stirring member 14 therein to stir the fermentation material in the cavity 11.

In the present embodiment, the first inlet 211, the second inlet 221, and the third inlet 312 are independent to each other, and the first outlet 212, the second outlet 222, and the third outlet 311 are also independent to each other. However, in other possible embodiments, the first inlet 211, the second inlet 221, and the third inlet 312 can converge into a same inlet, and the first outlet 212, the second outlet 222, and the third outlet 311 can also converge into a same outlet. Besides, the third inlets 312 of the plural flow disturbing plates 30 can converge into a same inlet, the third outlets 311 can also converge into a same outlet.

In use, the exterior pipe, the interior pipe, and the heat exchange channel are adapted for steam, hot water, or others to pass through for heating. Because the gaps of the exterior pipe don't contribute to the heating, the interior pipe can make up for it. Thus, the whole wall of the tank body can be heated evenly. In addition, the conventional flow disturbing plates are disposed with heat exchange channels, so the efficiency of heating is improved.

Besides, the interior pipe disposed on the inner wall of the tank body is easy to wash.

In conclusion, the fermentation tank has improved efficiency of heat exchanging, and the heat exchange is even. In addition, the heating device may not occupy too much space of the cavity. Furthermore, the fermentation tank is easy to wash.

What is claimed is:

1. A fermentation tank, characterized by including:
a tank body, having a top end and a bottom end and enclosing a cavity therein, a vertical direction being defined from a line composed of the top end and the bottom end, the tank body having a material inlet and a material outlet;
a heat exchange structure, disposed on a wall of the cavity for heat exchanging with the tank body;
at least one flow disturbing plate, the at least one flow disturbing plate being an elongated plate and having a width direction, a longitudinal direction, and a thickness direction which are perpendicular to each other, the width direction being parallel to a radial direction of the tank body, the longitudinal direction being parallel to the vertical direction, a length of the at least one flow disturbing plate being larger than a width thereof, the at least flow disturbing plate being arranged in the cavity and being connected to the tank body, the at least one flow disturbing plate having a heat exchange channel therein, two ends of the heat exchange channel communicating an exterior respectively so that heat is exchanged between the at least one flow disturbing plate and the cavity.

2. The fermentation tank of claim 1, wherein the heat exchange structure includes an exterior pipe, the exterior pipe extends as helix around an outer surface of the tank body, adjacent turns of the exterior pipe is spacedly arranged to form a gap therebetween, the exterior pipe has a first inlet and a first outlet, the first inlet and the first outlet communicate the exterior respectively.

3. The fermentation tank of claim 2, wherein the first inlet is located at a top of the exterior pipe, the first outlet is located at a bottom of the exterior pipe.

4. The fermentation tank of claim 2, wherein the first inlet is located at a bottom of the exterior pipe, the first outlet is located at a top of the exterior pipe.

5. The fermentation tank of claim 2, wherein the heat exchange structure further includes an interior pipe, the interior pipe extends as helix around an inner surface of the tank body, the interior pipe positionally corresponds to the gaps of the exterior pipe, the interior pipe has a second inlet and a second outlet, the second inlet and the second outlet communicate the exterior respectively.

6. The fermentation tank of claim 1, wherein the heat exchange channel extends the longitudinal direction and has a third inlet and a third outlet, the third inlet and the third outlet communicate the exterior respectively.

7. The fermentation tank of claim 6, wherein the heat exchange channel extends in a meandering way in the at least one flow disturbing plate, the third inlet is located at a bottom of the heat exchange channel, the third outlet is located at a top of the heat exchange channel.

8. The fermentation tank of claim 1, wherein the at least one flow disturbing plate includes at least four flow disturbing plates.

9. The fermentation tank of claim 1, wherein a stirring member is arranged in the tank body to stir fermentation material in the cavity.
